(19) United States
(12) Patent Application Publication
(10) Pub. No.: US 2002/0147196 A1
(43) Pub. Date: Oct. 10, 2002

(54) Composition and method for treating neuropathic pain

(76) Inventors: Steven Noel Quessy, Durham, NC (US); George David Rudd, Durham, NC (US); Michael Fredrik Winnem, Greenford, NC (US)

Correspondence Address: DAVID J LEVY, CORPORATE INTELLECTUAL PROPERTY, GLAXOSMITHKLINE, FIVE MOORE DR., PO BOX 13398, RESEARCH TRIANGLE PARK, NC, 27709-3398, US

(21) Appl. No.: 10/115,548

(22) Filed: Apr. 3, 2002

Related U.S. Application Data
(60) Provisional application No. 60/281,635, filed on Apr. 5, 2001.

(51) Int. Cl. A61K 31/537; A61K 31/55; A61K 31/53

(52) U.S. Cl. 514/231.2; 514/217; 514/245; 514/390

(57) ABSTRACT

This invention relates to a composition and method for alleviating neuropathic pain and/or its symptoms. The composition comprises (1) a compound that inhibits the reuptake of both norepinephrine and dopamine or inhibits the reuptake of norepinephrine alone in combination with (2) a compound that acts as a sodium channel blocker. The method involves the administration of the composition in an effective amount to alleviate neuropathic pain and/or its symptoms.

### Field of the Invention

This invention relates to a composition and method for alleviating neuropathic pain. More particularly, the present invention relates to a composition comprising (1) a compound that inhibits the reuptake of both norepinephrine and dopamine or inhibits the reuptake of norepinephrine in combination with (2) a compound that acts as a sodium channel blocker. The invention also relates to the administration of said composition.

### BACKGROUND OF THE INVENTION

Neuropathic pain syndromes can develop following neuronal injury and the resulting pain may persist for months or years, even after the original injury has healed.

Neuronal injury may occur in the peripheral nerves, dorsal roots, spinal cord or certain regions in the brain.

Neuropathic pain syndromes are traditionally classified according to the disease or event that precipitated them. Such neuropathic pain syndromes include: diabetic neuropathy; sciatica; non-specific lower back pain; multiple sclerosis pin; fibromyalgia; HIV-related neuropathy; post-herpetic neuralgia; trigeminal neuralgia; and pain resulting from physical trauma, amputation, cancer, toxins or chronic inflammatory conditions. These conditions are difficult to treat and although several drugs are known to have limited efficacy, complete pain control is rarely achieved.

The symptoms of neuropathic pain are incredibly heterogeneous and are often described as spontaneous shooting and lancinating pain, or ongoing, burning pain. In addition, there is pain associated with normally non-painful sensations such as “pins and needles” (paraesthesias and dysesthesias), increased sensitivity to touch (hyperesthesia), painful sensation following innocuous stimulation (dynamic, static, or thermal allodynia), increased sensitivity to noxious stimuli (thermal, cold, mechanical hyperalgesia), continuing pain sensation after removal of the stimulation (hyperpathia) or an absence of or deficit in selective sensory pathways (hypoalgesia).

As can be seen, neuropathic pain can be described by a number or heterogeneous conditions. Such conditions neither can be explained by a single etiology nor by a particular anatomical lesion. However, despite a different etiology and multiple lesions giving rise to neuropathic pain, many of these conditions share common clinical phenomena such as no visible injury, paradox combination of sensory loss and hyperalgesia in a painful area, paroxysms and a gradual increase of pain following repetitive stimulation.

Neuropathic pain can occur at any age and is most prevalent in adults. It has been estimated that one percent of the human population has moderate to severe neuropathic pain and that this may be as high as fifty percent in the elderly. Accordingly, there is a need for a pharmaceutical composition that can alleviate neuropathic pain and/or its symptoms effectively and a method for administering such composition.

### SUMMARY OF THE INVENTION

Accordingly, there is provided a composition and method for alleviating neuropathic pain. The composition of the invention comprises (1) a compound that inhibits the reuptake of both norepinephrine and dopamine or inhibits the reuptake of norepinephrine, or a pharmaceutically acceptable derivative thereof in combination with (2) a compound that acts as a sodium channel blocker, or a pharmaceutically acceptable derivative thereof. The method comprises administering an effective amount of said composition, or each of the compounds comprising it, to alleviate neuropathic pain and/or its symptoms.

### DETAILED DESCRIPTION OF THE INVENTION

The composition of the invention comprises (1) a compound that inhibits the reuptake of both norepinephrine and dopamine (NE-DA reuptake inhibitor) or inhibits the reuptake of norepinephrine (NE reuptake inhibitor), or a pharmaceutically acceptable derivative thereof, in combination with (2) a compound that acts as a sodium channel blocker (Na+ channel blocker), or pharmaceutically acceptable derivative thereof. While not wishing to be limited or bound by any theory, it is believed that the NE-DA- and NE-reuptake inhibitors exert analgesic activity by affecting descending pain pathways. However, such compounds may be associated with dose-limiting side effects such as, for example, seizure, insomnia, and/or rash. On the other hand, it is believed that Na+ channel blockers, especially those used for the treatment of epilepsy, can be effective in the treatment of neuropathic pain in some patients because these channel blockers have analgesic efficacy affecting ascending pain pathways so as to block neuronal hyperexcitability peripherally. Therefore, the combination manifests synergism in at least one of the following: greater efficacy as measured by lower pain scores; efficacy in a greater number of patients; equivalent efficacy with the combination using lower doses of each agent; faster onset of action with the combination; lower incidence of undesirable side effects; fewer patients using additional medications to relieve symptoms, and/or longer lasting pain relief.

Accordingly, suitable for use as NE-DA reuptake inhibitors are bupropion with or without one or more stabilizers, (2S,3S,5R)-2-(3,5-difluorophenyl)-3,5-dimethyl-2-morpholinol, (2S,3S)-2-(3-chlorophenyl)-3,5,5-trimethyl-2-morpholinol, and (2R,3R)-2-(3-chlorophenyl)-3,5,5-trimethyl-2-morpholinol. Suitable NE reuptake inhibitors are nortripyline, desipramine, maprotiline, venlafaxine, amitriptyline, and imipramine. Collectively NE-DA reuptake inhibitors and NE reuptake inhibitors are referred to herein as Compound(s) 1. It is also understood that mixtures of these compounds can be employed in the invention. Preferred among these compounds are bupropion, (2S,3S,5R)-2-(3,5-difluorophenyl)-2-dimethyl-2-morpholinol, (2S,3S)-2-(3-chlorophenyl)-3,5,5-trimethyl -2-morpholinol, and (2R,3R)-2-(3-chlorophenyl)-3,5,5-trimethyl-2-morpholinol, nortripyline, and desipramine. Compounds such as bupropion, nortripyline, desipramine, maprotiline, venlafaxine, amitriptyline, and imipramine are known in the art and readily available. Bupropion is disclosed for example in U.S. Pat. Nos. 5,358,970 and RE 33,994. Bupropion is readily commercially available as Wellbutrin IR, Wellbutrin SR, and Zyban. The instant, sustained, and/or extended release forms of bupropion can be employed in the present invention. Preferably, bupropion is used as a salt (organic and/or inorganic salt) such as the hydrochloride (HCl) salt form. Stabilizers associated with bupropion are also well known and can be employed in the present invention. (2S,3S,5R)-2-(3,5-difluorophenyl)-3,5-dimethyl-2-morpholinol is disclosed, for example, in PCT/EP99/07117; EP0,426,416; U.S. Pat. No. 5,104,870; and PCT/US00/27252. (2S,3S)-2-(3-chlorophenyl)-3,5,5-trimethyl-2-morpholinol is disclosed in PCT/US99/01134. (2R,3R)-2-(3-chlorophenyl)-3,5,5-trimethyl-2-morpholinol is disclosed in at least one of the above-identified references.

Suitable for use as Na+ channel blockers, referred to herein as Compound(s) (2), are lamotrigine, oxcabazepine, carbamazepine, phenytoin, lidocaine, and 6-(2,3,5-trichlorophenyl) -1,2,4-triazin-5-ylamine. Channel blockers such as lamotrigine (Lamictal™), oxcarbazepine, carbamazepine, phenytoin, and lidocaine are well known and readily commercially available. 6-(2,3,5-trichlorophenyl)-1,2,4-triazin-5-ylamine is disclosed, for example, in PCT/EP98/08273. It is to be understood that mixtures of these Na+ channel blockers can be employed in the invention if so desired.

Administration. Compounds for use according to the invention may be administered simultaneously or sequentially and, when administration is sequential, either of the functional types of compounds may be administered first. When admisistration is simultaneous, the combination may be administered either in the same or different pharmaceutical composition.

Compounds for use according to the invention may be administered as the raw material but the active ingredients are preferably provided in the form of pharmaceutical formulations.

The active ingredients may be used either as separate formulations or as a single combined formulation. When combined in the same formulation it will be appreciated that the two compounds must be stable and compatible with each other and the other components of the formulation. Therefore, pharmaceutical formulations comprising a combination as defined herein together with a pharmaceutically acceptable diluent or carrier comprise a further aspect of the invention. When formulated separately they may be provided in any convenient formulation, in such manner known for such compounds in the art.

Accordingly in a further aspect of the invention there is provided a pharmaceutical composition which comprises (1) a compound that has a primary mechanism of inhibiting the reuptake of both norepinephrine and dopamine (or has a primary mechanism of inhibiting the reuptake of norepinephrine alone) including pharmaceutically acceptable derivatives thereof and (2) a sodium channel blocker including pharmaceutically acceptable derivatives thereof formulated for administration by any convenient route. Such compositions are preferably in a form adapted for use in medicine, in particular for mammalian medicine, especially human medicine, and can conveniently be formulated in conventional manner using one or more pharmaceutically acceptable carriers or excipients.

The formulations include those suitable for oral, parenteral (including subcutaneous, e.g., by injection or by depot tablet, intradermal, intrathecal, intramuscular, e.g., by depot and intravenous), rectal, topical (including dermal, buccal and sublingual), or in a form suitable for administration by inhalation or insufflation administration, although the most suitable route may depend upon, for example, the condition and disorder of the recipient. The formulations may conveniently be presented in unit dosage form and may be prepared by any of the methods well known in the art of pharmacy. In general, these methods include the step of bringing into association the compounds (“active ingredient”) with a carrier that constitutes one or more accessory ingredients. In general the formulations are prepared by uniformly and intimately bringing into association the active ingredient(s) with liquid carriers or finely divided solid carriers or both and then, if necessary, shaping the product into the desired formulation. Preferably such compositions will be formulated for oral administration. It will be appreciated that when two active ingredients are administered independently, each may be administered by different means.

Formulations suitable for oral administration may be presented as discrete units such as capsules, cachets or tablets (e.g., chewable tablet in particular for pediatric administration) each containing a predetermined amount of the active ingredient; as a powder or granules; as a solution or suspension in an aqueous liquid or a non-aqueous lilquid; or as an oil-in-water liquid emulsion or a water-in-oil liquid emulsion. The active ingredient may also be presented as a bolus, electuary or paste.

A tablet may be made by compression or molding, optionally with one or more accessory ingredients. Compressed tablets may be prepared by compressing in a suitable machine the active ingredient in a free-flowing form such as a powder or granules, optionally mixed with other conventional excipients such as binding agents, (for example, syrup, acacia, gelatin, sorbitol, trgacanth, mucilage of starch, polyvinylpyrrolidone) or hydroxymethyl cellulose or hydoxymethyl cellulose fillers (for example, lactose, sugar, microcrystalline cellulose, maize-starch, calcium phosphate or sorbitol), lubricants (for example, magnesium stearate, stearic acid, talc, polyethylene glycol or silica), disintegrants (for example, potato starch or sodium starch glycollate), wetting agents, such as sodium lauryl sulfate, or flavoring. Inert and/or inactive and/or non-bio-active (e.g., non-bioequivalent) forms of the compounds of the invention may also be considered as excipients for purposes of formulating. Molded tablets may be made by molding in a suitable machine a mixture of the powdered compound moistened with an inert liquid diluent. The tablets may optionally be coated or scored and may be formulated so as to provide slow or controlled release of the active ingredient therein. The tablets may be coated according to methods well-known in the art.

Alternatively, the compounds comprising the combination of the invention may be incorporated into oral liquid preparations such as aqueous or oily suspensions, solutions, emulsions, syrups or elixirs, for example. Moreover, formulations of the invention may be presented as a dry product for constitution with water or other suitable vehicle before use. Such liquid preparations may contain conventional additives such as suspending agents such as sorbitol syrup, methyl cellulose, glucose/sugar syrup, gelatin, hydroxyethylcellulose, carboxymethyl cellulose, aluminum stearate gel or hydrogenated edible fats; emulsifying agents such as lecithin, sorbitan mono-oleate or acacia; non-aqueous vehicles (which may include edible oils) such as almond oil, fractionated coconut oil, oily esters, propylene glycol or ethyl alcohol; preservatives such as methyl or propyl p-hydroxybenzoates or sorbic acid, and flavoring.

Preparations may also be formulated as suppositories, e.g., containing conventional suppository bases such as cocoa butter, hard fat, polyethylene glycol, or other glycerides.

Formulations for parenteral administration include aqueous and non-aqueous sterile injection solutions which may contain anti-oxidants, buffers, bacteriostats, and solutes which render the formulation isotonic with the blood of the intended recipient; and aqueous and non-aqueous sterile suspensions which may include suspending agents and thickening agents.

The formulations may be presented in unit-dose or multi-dose containers, for example, sealed ampoules and vials, and may be stored in a freeze-dried (lyophilised) condition requiring only the addition or a sterile liquid carrier, for example, water for injection, immediately prior to use. Extemporaneous injection solutions and suspensions may be prepared form sterile powders, granules and tablets of the kind previously described.

For topical administration in the mouth, for example, bucally or sublingually, include lozenges comprising the active ingredient in a flavored basis such as sucrose and acacia or tragacanth, and pastilles comprising the active ingredient in a basis such as gelatin and glycerin or sucrose and acacia.

For topical administration to the epidermis, the compounds may be formulated as creams, gels, ointments, lotions, or as a transdermal patch.

The invention may be formulated as depot preparations. Such long-acting formulations may be administered by implantation (for example, subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

For intranasal administration the compounds and/or compositions of the invention may be used, for example as a liquid spray, as a powder or in the form of drops.

Administration of the compounds/compositions of the invention can be accomplished by delivery in the form of an aerosol spray presentation from pressurized packs or a nebuliser, with the use of a suitable propellant, e.g., 1,1,1,2-trifluoroethane (HFA 134A) and 1,1,1,2,3,3,3-heptapropane (HFA 227), carbon dioxide or other suitable gas. In the case of pressurized aerosol the dosage unit may be determined by providing a valve to deliver a metered amount. Capsules and cartridges of, for example, gelatin for use in an inhaler or insufflator may be formulated containing a powder mix of the compound or composition of the invention and a suitable powder base such as lactose or starch.

Dosage. It will be appreciated by those skilled in the art that treatment extends to prophylaxis as well as the treatment of the established disease or symptom(s). Moreover, it will be appreciated that the amount of a compound/composition of the invention required for use in the treatment will vary with the nature of the condition being treated, the age and condition of the patient, and will be ultimately at the discretion of the attendant physician or veterinarian. In general, doses employed for adult human (e.g., 70 kg body weight) treatment will typically be in the range of 10 to 350 mg per day, preferably 25 to 300 mg, most preferably 75 to 300 mg per day for compound (1) of the composition and in the range of 5 to 1200 mg per day, preferably 100 to 650 mg, most preferably 200 to 400 mg per day for compound (2) of the composition.

Pharmaceutical compositions according to the invention may be prepared by conventional techniques. When combined in the same formulation, for example, compounds (1) and (2) or a pharmaceutically acceptable derivatives thereof may be admixed together, if desired, with suitable excipients. Tablets may be prepared, for example, by direct compression of such a mixture. Capsules may be prepared, for example, by filling a blending of the compounds together with suitable excipients into gelatin capsules using a suitable filling machine.

Compositions for use according to the invention may, if desired, be presented in a pack or dispenser device, which may contain one or more unit dosage forms containing the active compounds. The pack may, for example, comprise metal or plastic foil, such as a blister pack. Where compounds are intended for administration as two separate compositions these may be presented, for example, in the form of a twin pack.

Pharmaceutical compositions of the invention may also be prescribed to the patient in “patient packs” containing the whole course of treatment in a single package, usually a blister pack. Patient packs have an advantage over traditional prescriptions, where a pharmacists divides a patient's supply of a pharmaceutical from a bulk supply, in that the patient always has access to the package insert contained in the patient pack, normally missing in traditional prescriptions. The inclusion of a package insert has been shown to improve patient compliance with the physician's instructions. A patient pack is a desirable additional feature of the invention.

As used herein, the term “effective amount” means that amount of a drug or pharmaceutical agent that will elicit the biological or medical response of a tissue, system, animal or human that is being sought, for instance, by a researcher or clinician. Furthermore, the term “therapeutically effective amount” means any amount which, as compared to a corresponding subject who has not received such amount, results in improved treatment, healing, prevention, or amelioration of a disease, disorder, or side effect, or a decrease in the rate of advancement of a disease or disorder. The term also includes within its scope amounts effective to enhance normal physiological function.

As used herein, the term “physiologically functional derivative(s)” or “pharmaceutically acceptable derivative(s)” refers to any pharmaceutically acceptable derivative of a compound of the present invention, for example, an ester or an amide, which upon administration to a mammal is capable of providing (directly or indirectly) a compound of the present invention or an active metabolite thereof. Such derivatives are clear to those skilled in the art, without undue experimentation, and with reference to the teaching of Burger's Medicinal Chemistry And Drug Discovery, 5^{th} Edition, Vol. 1: Principles and Practice, which is incorporated herein by reference to the extent that it teaches physiologically functional derivatives.

Certain of the compounds of the invention described herein contain one or more chiral atoms, or may otherwise be capable of existing as two enantiomers. The compounds of this invention include mixtures of enantiomers as well as purified enantiomers or enantiomerically enriched mixtures. Also included within the scope of the invention are the individual isomers of the compounds represented by compounds (1) and (2) of the present invention as well as any wholly or partially equilibrated mixtures thereof. The present invention also covers the individual isomers of the compounds represented by the formulas above as mixtures with isomers thereof in which one or more chiral centers are inverted.

Typically, the salts of the present invention are pharmaceutically acceptable salts. Salts encompassed within the term “pharmaceutically acceptable salts” refer to non-toxic salts of the compounds of this invention. Salts of the compounds of the present invention may comprise salts of organic and inorganic acids. Representative salts include the following salts: acetate, benzenesulfonate, benzoate, bicarbonate, bisulfate, bitartrate, borate, bromide, calcium edetate, camsylate, carbonate, chloride, clavulanate, citrate, dihydrochloride, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isethionate, lactate, lactobionate, laurate, malate, maleate, mandelate, mesylate, methylbromide, methylnitrate, methylsulfate, monopotassium maleate, mucate, napsylate, nitrate, N-methylglucamine, oxalate, pamoate (embonate), palmitate, pantothenate, phosphate/diphosphate, polygalacturonate, potassium, salicylate, sodium, stearate, subacetate, succinate, tannate, tartrate, teoclate, tosylate, triethiodide, trimethylammonium and valerate. Other salts, which are not pharmaceutically acceptable, may be useful in the preparation of compounds of this invention and these form a further aspect of the invention.

### EXPERIMENTAL

### Test Procedure for Pre-clinical Experiments 1, 2 and 3

All experiments were performed using the chronic constriction injury (CCI) model of neuropathic pain in the rat. Under isoflurane anaesthesia, the common left sciatic nerve of male random hooded rats (180-220 g) was exposed at mid thigh level. Four ligatures of chromic gut (4.0) were tied loosely around the nerve with a 1 mm spacing between each. The wound was then closed and secured with suture clips. The surgical procedure was identical for the sham operated animals except the sciatic nerve was not ligated. The animals were allowed a period of 10 days to recover from the surgery before any behavioural testing began. The effect of the test compound on CCI-induced decrease in mechanical paw withdrawal threshold was measured using an algesymeter (Randall & Selitto, 1957). The presence of mechanical allodynia was assessed using Von Frey hair mono filaments (range: 4.19-84.96 g). The rats were lightly restrained and placed upon a metal grid floor, from below which the monofilaments were applied to the plantar surface of the hindpaws. The lowest monofilament to produce a withdrawal was the response recorded.

All data were expressed as mean±s.e.mean. Statistical analysis was carried out to compare the difference between the drug treated group and vehicle treated groups using unpaired Student's t-test (P<0.05 considered significant).

### Pre-clinical Experiment 1

### Test Compound

Lamotrigine (30 mgkg⁻¹ b.i.d. p.o.) was dosed chronically for a period of 12 days. The dose chosen was determined from previous “in-house” data and doses believed to be efficacious in the clinical treatment of neuropathic pain. No adverse side effects were observed as a result of the surgery or chronic dosing schedule.

### Results

Prior to the start of treatment, the CCI-operated animals were showing the characteristic decrease in paw withdrawal threshold, demonstrating the onset of neuropathic hypersensitivity. Chronic dosing with lamotrigine (30 mgkg⁻¹, twice daily, days 13-24 post-operative) by the oral route fully reversed (by day 16) the CCI-induced decrease in paw-withdrawal threshold to that of the sham-operated animals. The analgesic effect of lamotrigine appeared to be maintained following cessation of the treatment, until day 37 post-operative.

It was clear at the beginning of the experiment that the characteristic decrease in Von Frey hair withdrawal threshold was evident demonstrating the presence of neuropathic hypersensitivity in the rats. Chronic dosing with lamotrigine (30 mgkg⁻¹ b.i.d. p.o.) appeared to produce a transient reversal of the CCI-induced tactile allodynia. Following cessation of the treatment it appeared that the lamotrigine treated group showed a small reversal of the tactile allodynia compared to the vehicle treated group.

### Conclusion

Chronic dosing with lamotrigine reversed the CCI-induced decrease in paw withdrawal threshold back to sham operated levels. This reversal was maintained for several days following cessation of the treatment. It also appeared to have a transient effect on the CCI-induced tactile allodynia during and following cessation of treatment.

### Pre-clinical Experiment 2

### Test Compound

Bupropion (10 mgkg⁻¹ b.i.d. p.o.) was dosed chronically for a period of 8 days. The dose chosen reflects both previous “in-house” data and that which is believed to be efficacious in the clinical treatment of neuropathic pain. No adverse side effects were observed as a result of the surgery or chronic dosing schedule.

### Results

Prior to the start of treatment, the CCI-operated animals were showing the characteristic decrease in paw withdrawal threshold, demonstrating the onset of neuropathic hypersensitivity. A single dose of bupropion (10 mgkg⁻¹ ) produced an immediate reversal of the fall in paw withdrawal threshold back towards sham-operated levels. This reversal was maintained throughout the chronic dosing period with bupropion (10 mgkg⁻¹ b.i.d. p.o.; days 47-54), and for 4 days following cessation of the treatment. On the first day of dosing only, bupropion also had a small effect on paw withdrawal thresholds in sham-operated rats 1 hour post dose, suggesting a possible antinociceptive component in addition to the antihypersensitivity action.

It was clear before the beginning of the dosing period that the characteristic decrease in Von Frey hair withdrawal threshold was evident demonstrating the presence of neuropathic hypersensitivity in the rats. Chronic dosing with bupropion (10 mgkg⁻¹ b.i.d. p.o.; days 47-54) appeared to have some effect on the tactile allodynia, with a maximal reversal being achieved on the last day of dosing (day 54). This reversal was not maintained following cessation of the treatment.

### Conclusion

Bupropion reversed the CCI-induced decrease in paw withdrawal threshold back to sham operated levels following a single dose. This reversal was maintained for the duration of the dosing period and for 4 days following cessation of the treatment. Bupropion also appeared to have some effect on the CCI-induced tactile allodynia producing a maximal reversal on the last day of dosing.

### Pre-clinical Experiment 3

### Test Compounds

For the combination (lamotrigine and bupropion) study two doses are chosen:

(1) the minimum effective dose shown previously to have efficacy in the CCI model

(2) doses lower than those shown previously to be effective in the model. The animals are monitored for side effects as a result of either the surgery or dosing schedule, substantially as set forth in the previous experiments, except for doses.

### Results

The presence of neuropathic hypersensitivity is assessed as a characteristic decrease in paw withdrawal threshold in the CCI-operated animals when compared to the sham-operated animals. Once a stable baseline of hypersensitivity is achieved chronic dosing is commenced. The combination of lamotrigine and bupropion is dosed chronically for a period of 10 days. One group of animals received the known minimum effective dose of each compound in the CCI model (10 mgkg⁻¹ b.i.d. p.o.). The other group of animals received a lower dose of each compound unlikely to be efficacious in its own right (3 mgkg⁻¹ b.i.d. p.o.). The effect of each combination treatment on the paw withdrawal threshold of the animals is examined 1, 3 and 5 hours post a single dose and then 3 hours post-dose on each of the subsequent days during chronic dosing and after cessation of treatment.

Once it is clear that the characteristic decrease in Von Frey hair withdrawal threshold is evident demonstrating the presence of neuropathic hypersensitivity in the rats, chronic dosing is commenced. The withdrawal thresholds for the two groups receiving lamotrigine and bupropion in combination (3 mgkg⁻¹ & 10 mgkg⁻¹ b.i.d. p.o.) is assessed 1, 3 and 5 hours post a single dose and then 3 hours post-dose on each of the subsequent days during chronic dosing and after cessation of treatment.

### Conclusion

The combination of lamotrigine and bupropion demonstrates a synergy using the two compounds. This may be evident as any or all of the following:

greater efficacy against paw withdrawal threshold or von Frey monofilaments in individual rats; efficacy in a greater number of rats; equivalent efficacy with the combination using lower doses of each agent; faster onset of action with the combination; or longer lasting pain relief.

What is claimed is:

1. A pharmaceutical composition comprising: (1) a compound that inhibits the reuptake of both norepinephrine and dopamine or inhibits the reuptake of norepinephrine alone, or pharmaceutically acceptable derivative thereof, in combination with (2) a compound that acts as a sodium channel blocker, or pharmaceutically acceptable derivative thereof.

2. The pharmaceutical composition of claim 1 wherein the compound that inhibits the reuptake of both norepinephrine and dopamine or inhibits the reuptake of norepinephrine alone is selected from the group consisting of bupropion, (2S,3S,5R)-2-(3,5-difluorophenyl) -3,5-dimethyl-2-morpholinol, (2S,3S)-2-(3-chlorophenyl)-3,5,5-trimethyl -2-morpholinol, (2R,3R)-2-(-3-chlorophenyl)-3,5,5-trimethyl-2-morpholinol, nortriptyline, desipramine, maprotiline, venlafaxine, amitriptyline, imipramine, and mixtures thereof.

3. The pharmaceutical composition of claim 1 wherein the compound that acts as a sodium channel blocker is selected from the group consisting of lamotrigine, oxcarbazepine, carbamazepine, phenytoin, 6-(2,3,5-trichlorophenyl)-1,2,4-triazin-5-ylamine, lidocaine, and mixtures thereof.

4. The pharmaceutical composition of claim 1 wherein the compound that inhibits the reuptake of both norepinephrine and dopamine or inhibits the reuptake of norepinephrine alone is selected from the group consisting of bupropion, (2S,3S,5R)-2-(3,5-difluorophenyl)-3,5-dimethyl-2-morpholinol, (2S,3S)-2-(3-chlorophenyl)-3,5,5-trimethyl-2-morpholinol, (2R,3R)-2-(-3-chlorophenyl)-3,5,5-trimethyl-2-morpholinol, and mixtures thereof.

5. The pharmaceutical composition of claim 1 wherein the compound that acts as a sodium channel blocker is selected from the group consisting of lamotrigine, 6-(2,3,5-trichlorophenyl)-1,2,4-triazin-5-ylamine, and mixtures thereof.

6. The pharmaceutical composition of claim 1 wherein the compound that inhibits the reuptake of both norepinephrine and dopamine or inhibits the reuptake of norepinephrine alone is bupropion and the compound that acts as a sodium channel blocker is lamotrigine.

7. The pharmaceutical composition of claim 1 wherein the compound that inhibits the reuptake of both norepinephrine and dopamine or inhibits the reuptake of norepinephrine alone is (2S,3S,5R)-2-(3,5-difluorophenyl)-3,5-dimethyl-2-morpholinol and the compound that acts as a sodium channel blocker is lamotrigine.

8. The pharmaceutical composition of claim 1 wherein the compound that inhibits the reuptake of both norepinephrine and dopamine or inhibits the reuptake of norepinephrine alone is (2S,3S)-2-(3-chlorophenyl)-3,5,5-trimethyl-2-morpholinol and the compound that acts as a sodium channel blocker is lamotrigine.

9. The pharmaceutical composition of claim 1 wherein the compound that inhibits the reuptake of both norepinephrine and dopamine or inhibits the reuptake of norepinephrine alone is (2R,3R)-2-(-3-chlorophenyl)-3,5,5-trimethyl-2-morpholinol and the compound that acts as a sodium channel blocker is lamotrigine.

10. The pharmaceutical composition of claim 1 wherein the compound that inhibits the reuptake of both norepinephrine and dopamine or inhibits the reuptake of norepinephrine alone is bupropion and the compound that acts as a sodium channel blocker is 6-(2,3,5-trichlorophenyl)- 1,2,4-triazin-5-ylamin.

11. The pharmaceutical composition of claim 1 wherein the compound that inhibits the reuptake of both norepinephrine and dopamine or inhibits the reuptake of norepinephrine alone is (2S,3S,5R)-2-(3,5-difluorophenyl)-3,5-dimethyl-2-morpholinol and the compound that acts as a sodium channel blocker is 6-(2,3,5-trichlorophenyl)-1,2,4-triazin-5-ylamin.

12. The pharmaceutical composition of claim 1 wherein the compound that inhibits the reuptake of both norepinephrine and dopamine or inhibits the reuptake of norepinephrine alone is (2S,3S)-2-(3-chlorophenyl)-3,5,5-trimethyl-2-morpholinol and the compound that acts as a sodium channel blocker is 6-(2,3,5-trichlorophenyl)-1,2,4-triazin-5-ylamin.

13. The pharmaceutical composition of claim 1 wherein the compound that inhibits the reuptake of both norepinephrine and dopamine or inhibits the reuptake of norepinephrine alone is (2R,3R)-2-(-3-chlorophenyl)-3,5,5-trimethyl-2-morpholinol and the compound that acts as a sodium channel blocker is 6-(2,3,5-trichlorophenyl)-1,2,4-triazin-5-ylamin.

14. A method for the treatment of a mammal suffering from neuropathic pain comprising the administration of an effective amount of a composition which comprises (1) a compound that inhibits the reuptake of both norepinephrine and dopamine or inhibits the reuptake of norepinephrine alone, or pharmaceutically acceptable derivative thereof, in combination with (2) a compound that acts as a sodium channel blocker, or pharmaceutically acceptable derivative thereof.

15. The method of claim 14 wherein the compound that inhibits the reuptake of both norepinephrine and dopamine or inhibits the reuptake of norepinephrine alone is selected from the group consisting of bupropion, (2S,3S,5R)-2-(3,5-difluorophenyl)-3.5-dimethyl -2-morpholinol, (2S,3S)-2-(3-chlorophenyl)-3,5,5-trimethyl-2-morpholinol, (2R,3R)-2-(-3-chlorophenyl)-3,5,5-trimethyl-2-morpholinol, nortriptyline, desipramine, maprotiline, venlafaxine, amitriptyline, imipramine, and mixtures thereof.

16. The method of claim 14 wherein the compound that acts as a sodium channel blocker is selected from the group consisting of lamotrigine, oxcarbazepine, carbamazepine, phenytoin, 6-(2,3,5-trichlorophenyl)-1,2,4-triazin-5-ylamine, lidocaine, and mixtures thereof.

17. The method of claim 14 wherein the compound that inhibits the reuptake of both norepinephrine and dopamine or inhibits the reuptake of norepinephrine alone is selected from the group consisting of bupropion, (2S,3S,5R)-2-(3,5-difluorophenyl)-3,5-dimethyl -2-morpholinol, (2S,3S)-2-(3-chlorophenyl)-3,5,5-trimethyl-2-morpholinol, (2R,3R)-2-(-3-chlorophenyl)-3,5,5-trimethyl-2-morpholinol, and mixtures thereof; and the compound that acts as a sodium channel blocker is selected from the group consisting of lamotrigine, 6-(2,3,5-trichlorophenyl)- 1,2,4-triazin-5-ylamine, and mixtures thereof.

18. The method of claim 14 wherein the compound that inhibits the reuptake of both norepinephrine and dopamine or inhibits the reuptake of norepinephrine alone is bupropion and the compound that acts as a sodium channel blocker is lamotrigine.

19. The method according to claim 14 wherein the composition is administered orally, sublingually, rectally, subcutaneously, or intraveneously.

20. The method according to claim 14 wherein the composition is administered as a tablet or suspension.

21. The pharmaceutical composition of claim 4 wherein bupropion is employed in salt form and a stabilizer is present.

22. The pharmaceutical composition of claim 4 wherein bupropion is employed in an instant release, sustained release, or extended release formulation.
